(19) United States
(12) Patent Application Publication
(10) Pub. No.: US 2014/0316534 A1
(43) Pub. Date: Oct. 23, 2014

(54) STENT

(71) Applicant: Photocure ASA, Oslo (NO)

(72) Inventors: Ketil Widerberg, Oslo (NO); Bjørn Klem, Oslo (NO)

(21) Appl. No.: 14/351,478

(22) PCT Filed: Oct. 12, 2012
(86) PCT No.: PCT/EP2012/070294

§ 371 Date: Apr. 11, 2014

(30) Foreign Application Priority Data
Oct. 14, 2011 (EP) 11185253.9

(51) Int. Cl. A61F 2/04; A61B 5/00

(52) U.S. Cl. CPC A61F 2/04; A61B 5/0071; A61F 2002/044
USPC 623/23.7; 600/476; 427/2.25

(57) ABSTRACT

This invention relates to the treatment and diagnosis of abnormalities of the epithelial-lined surface of the esophagus and, in particular, to esophageal stents having a surface coating comprising a photosensitizing agent or a precursor thereof for use in methods of photodynamic treatment (PDT) or photodynamic diagnosis (PDD) of the esophagus. The stents find particular use in methods of diagnosing and/or treating Barrett's esophagus and in diagnostic methods for monitoring the progression of the disease.

This invention relates generally to the treatment and diagnosis of abnormalities of the epithelial-lined surface of the esophagus.

More specifically, it relates to esophageal stents for use in delivering active agents which are photosensitising agents (or precursors thereof) to the esophagus. These find particular use in methods of photodynamic treatment (PDT) or photodynamic diagnosis (PDD) of the esophagus, for example in methods of diagnosing and/or treating Barrett's esophagus and in diagnostic methods for monitoring the progression of the disease.

Photodynamic treatment (PDT) is a relatively new technique for the treatment of pre-cancerous tissue abnormalities, cancerous tissue and non-cancerous diseases. PDT involves the administration of a photosensitiser or a precursor thereof to an area of interest. The photosensitiser or precursor thereof is taken up into the cells, where a precursor of a photosensitiser is converted into a photosensitiser. Upon exposure of the area of interest to light, the photosensitiser is excited, usually from a ground singlet state to an excited singlet state. It then undergoes intersystem crossing to a longer-lived excited triplet state. One of the few chemical species present in tissue with a ground triplet state is molecular oxygen. When the photosensitiser and an oxygen molecule are in proximity, an energy transfer can take place that allows the photosensitiser to relax to its ground singlet state, and create an excited singlet state oxygen molecule. Singlet oxygen is a very aggressive chemical species and will very rapidly react with any nearby biomolecules. Ultimately, these destructive reactions will kill cells for example through apoptosis or necrosis, whereby for instance cancer cells are selectively killed. The mechanisms are still not fully understood, but it is suggested that the clinical result, such as the selectivity for e.g. cancerous tissue, is not due to selective uptake by cancerous cells. Rather, there are similar levels of uptake in all cell types, but the processes of conversion and elimination are different in metabolically active cells, such as cancerous cells and also dysplastic or neoplastic cells (pre-cancerous abnormal cells), inflamed or infected cells, leading to a concentration gradient between such cells and normal tissue.

Photodynamic diagnosis (PDD) is similarly based on the principle that the photosensitiser, or a precursor of the photosensitiser, accumulates preferentially in metabolically active tissue, such as dysplastic or cancerous tissue. Upon photoactivation, i.e. exposure of the area of interest to light, the photosensitiser is excited and displays in response a fluorescence which is detected. Using this technique, cancerous tissues can be distinguished from healthy tissue.

Esophageal cancer includes both squamous cell carcinoma and adenocarcinoma. The incidence of esophageal adenocarcinoma has increased by almost 400% during recent decades and it is now the cancer with the fastest rising incidence in the Western world. Although it can be treated, for example by surgery or chemotherapy, it is rare that the disease can be cured; the mortality rate for those diagnosed with the disease is over 85%. Early diagnosis is therefore critical in order to improve survival rates.

Most esophageal adenocarcinomas are considered to arise from the condition known as ‘Barrett's esophagus’. Barrett's esophagus is a condition in which the normal squamous lining of the distal esophagus is replaced by columnar epithelium with intestinal metaplasia. It may be recognised endoscopically by a salmon-coloured mucosa above the lower esophageal sphincter that separates the stomach from the esophagus (a normal esophagus is usually light pink in colour). The condition typically arises as a complication of gastro-esophageal reflux disorder (GERD) which causes the normal cells that line the esophagus (squamous cells) to turn into specialised columnar cells that resemble those found in the intestinal epithelium (‘intestinal metaplasia’).

Intestinal metaplasia is a pre-malignant condition that, through a series of cellular changes, may develop further into low-grade dysplasia, high-grade dysplasia (pre-cancerous tissue abnormalities) and, ultimately, adenocarcinoma of the esophagus. Dysplasia is not usually visible when using standard endoscopic surveillance methods and can remain undetected until the invasive adenocarcinoma stage. The ability to monitor the various stages of the disease is further complicated by the simultaneous presence of metaplasia as well as dysplasia and early cancer which are difficult to differentiate with standard endoscopy techniques. The ability not only to diagnose Barrett's esophagus at an early stage, but also to monitor its progression is key to preventing esophageal cancer and to improving survival rates.

Currently, Barrett's esophagus is diagnosed and its progression monitored by taking random four-quadrant biopsies (RFQB) for every 1-2 cm length of the esophagus followed by histopathologic assessment of the biopsy specimens (Bartelsman et al. Eur. J. Cancer Prevention, 1: 323-325, 1992). Patients with known intestinal metaplasia undergo regular follow-up endoscopy with RFQB. This procedure increases the rate of early diagnosis of adenocarcinomas, but investigates only a small proportion of the epithelial surface which may be at risk; it is therefore associated with sampling errors (note that dysplastic changes often occur in a spatially heterogeneous fashion). Other drawbacks are that the method is costly, time-consuming to perform, and generally uncomfortable for the patient.

Alternative techniques for improved detection of intraepithelial dysplasia in Barrett's esophagus have been proposed. Endoscopic fluorescence detection following administration and photoactivation (i.e. activation with light) of 5-aminolevulinic acid (5-ALA) has been investigated. After systemic administration of 5-ALA and uptake into cells, it is converted intracellularly to the photosensitiser protoporphyrin IX (PpIX). Upon photoactivation, i.e. exposure of the area of interest to light, PpIX is excited and displays in response a fluorescence which is detected.

Studies have been carried out to assess the performance of 5-ALA in endoscopic fluorescence detection of intraepithelial dysplasia and early stage cancers in Barrett's esophagus (Stepinac et al., Endoscopy 35(8): 663-668, 2003). In these studies, systemic application of higher doses of 5-ALA at 20 mg/kg and 30 mg/kg was found to increase the sensitivity for detection of dysplastic tissue. However, specificity was found to be poor under these conditions (i.e. there was a high rate of false positive fluorescence). Systemic application of 20 mg/kg or more of 5-ALA is thus not recommended due to low specificity and increasing side effects at high dosages. It has been suggested that the high rate of false positives found in these earlier studies mainly arises due to the higher drug dose which induces a disturbing background fluorescence (i.e. noise) from inflamed, albeit otherwise healthy, mucosal tissues. Other limiting factors responsible for false positive fluorescence when using 5-ALA include the presence of metaplastic tissue which also induces strong fluorescence.

Local administration of formulations to the esophageal surface generally enables the use of lower dosages of the active photosensitising agent (or precursor thereof), thereby addressing some of the problems associated with systemic application. However, existing methods for topical application of formulations to the mucosa-lined surface of the esophagus have their drawbacks.

In WO 2009/109569, certain routes of administration of 5-ALA esters are proposed in methods of photodynamic imaging of esophageal cancer and Barrett's esophagus. These include topical delivery of aqueous solutions via a catheter and the use of sodium alginate compositions which coat the esophageal wall. Such methods involving the local delivery of solutions containing the active agent and the application of gels are, however, cumbersome and time-consuming. These can also result in a low drug contact time with the target tissues due to the tendency of the formulations to travel further down the esophagus and away from the application site; this reduces uptake of the drug by the target tissues. In the situation where formulations migrate further down the esophagus following local administration, this can also result in the delivery of surplus drug to the patient's stomach, e.g. particularly when using aqueous formulations.

There thus remains a need for alternative methods for the delivery of active photosensitising agents to the esophagus when carrying out methods of photodynamic therapy or diagnosis, especially when performing such methods for detecting and monitoring abnormal changes of the epithelial cells in Barrett's esophagus. The present invention provides a drug-coated esophageal stent which fulfills this need.

Stents for use in delivering therapeutic agents, including photosensitive agents, to a body lumen have been described previously, however these have been developed for very different applications to the present invention. Primarily, these are described for use in treating conditions within the vascular system where the stent serves to maintain the desired shape of the vessel, e.g. by opening or maintaining a passageway for the flow of blood. In such cases, the therapeutic agent is chosen for its ability to prevent or reduce any constrictive intraluminal disease such as restenosis.

For example, WO 2007/030478 describes a stent coated with an energy-activatable agent for long-term implantation into vessels to prevent obstruction of the vessel (e.g. by breaking down atherosclerotic plaques in arteries), the release of the active agent being controlled by a light stimulus which may be delivered to the patient after an extended time period.

Similarly, WO 94/15583 describes a slow-release drug-eluting stent for treatment of atherosclerosis, in particular restenosis of blood vessels following treatment of atherosclerotic plaques, by destroying the proliferating smooth muscle cells lining the vessel using a photosensitive agent and so preventing further obstruction. The purpose of this device is to maintain patency of vessels both by its physical presence in the vessel and by delivering active agents to breakdown proliferating cardiovascular tissue and thereby prevent flow obstruction. Such a device is not suitable for use in the esophagus, and does not address the problem of providing an alternative method for the controlled delivery of photosensitive agents for use in the diagnosis and treatment of esophageal abnormalities such as Barrett's esophagus.

Viewed from one aspect, the invention provides an esophageal stent comprising a generally cylindrical stent body having a surface coating comprising a photosensitising agent or a precursor thereof (also referred to herein as the “active agent”).

As used herein, the term “photosensitising agent” refers to agents which are photosensitive. It includes agents which on application of photoactivating light are converted to a cytotoxic form or give rise to a cytotoxic species (e.g. singlet oxygen). Further encompassed within the scope of this term are agents which on application of photoactivating light become excited and emit fluorescence which can be detected. A “precursor of a photosensitising agent” is an agent which in vivo is converted into a photosensitising agent as herein defined and which is thus essentially equivalent thereto (e.g. 5-ALA and 5-ALA esters).

Stents, including those suitable for use in the esophagus, are generally known and used in the art and may take a variety of forms. Any known type of stent which is suitable for deployment in the esophagus may be employed in the invention. The underlying structure (i.e. framework) of the stent body is not particularly limiting provided that this is capable of delivery to the esophagus and can be held in place (e.g. following radial expansion) to allow for contact with the surface tissue of the esophagus and delivery of the active agent.

The stent may be of the self-expandable type or the balloon-expandable type and will typically comprise an open framework onto which the active agent can be applied. The framework will generally be such that the stent can be delivered in a collapsed state (e.g. crimped onto a catheter) to enable this to pass safely down the esophagus before being expanded at the deployment site. Once in position, the stent can then be expanded, e.g. by inflation of a dilation balloon.

The stent body can be made of any material which is acceptable for introduction into the body. For example, this may be made of any bio-compatible material having the necessary physical characteristics for the chosen design of stent. Suitable stent materials are those which will not irritate the esophageal wall following deployment or cause any adverse reaction and include metals and polymers.

Examples of suitable metals include stainless steel, tantalum, cobalt alloys (e.g. cobalt-chromium alloys), platinum alloys (e.g. platinum-iridium alloys), and nickel alloys (e.g. nickel-titanium alloys).

Polymeric stents may be made from biostable or biodegradable polymer materials. Those made from biostable materials will generally be removed once the active agent has been delivered to the esophageal wall or, dependent on their light transmission characteristics, after PDT or PDD has been carried out. Biodegradable polymers are those which degrade or dissolve in the body. These have the advantage that they may not need to be removed from the esophagus following deployment, especially if these are transparent to light and so may be left in place during the PDT or PDD procedure. Depending on the nature of the polymer material, in some cases these may degrade before photoactivation is carried out.

Suitable biodegradable polymeric materials may be selected from the group consisting of poly-lactic acids, poly-glycolic acids, collagen, polycaprolactone, hyaluronic acid, polydioxanone, polycaprolactone, polygluconate, poly-lactic acid-polyethylene oxide copolymers, poly(hydroxybutyrate), polyanhydrides, polyphosphoesters, poly(amino acids), and poly(alpha-hydroxy acids). The nature of the biodegradable polymer may be chosen depending on the desired absorption rate.

Biostable polymers may also be used to form the stent body. Such materials include silicones, poly(ethylene terephthalate) and polyacetals.

Polymer-coated metals may also be used to form the stent body. These may comprise any combination of the polymer materials and metals hereinbefore described.

The stent body will typically comprise a framework or mesh made from a plurality of filaments or ‘struts’ which may be interwoven or inter-linked. Often the filaments will form a zig-zag or sinusoidal wave structure which enables the stent to be radially expanded from a contracted state. Depending on the precise geometry of the stent filaments, the stent coating which carries the active agent will generally cover the entire stent surface, i.e. both the inner and outer surfaces. However, in some cases, the stent coating will only be applied to the outer (i.e. tissue-contacting) surface. The coating may consist of a single coating or multiple coatings. Where multiple coatings are present, one or more of the individual layers may contain the active agent, preferably all layers. In some cases, it may, however, be desirable to apply an outer protective layer which need not necessarily contain any active agent.

The size of the stent may readily be selected according to need and will depend on factors such as the age and size of the patient to be treated, the extent of the abnormality to be treated or diagnosed, etc. The stent diameter should be such that, in use (i.e. in the expanded state), its outer surface contacts the esophagus wall such that the active agent can be delivered by contact transfer. The length of the stent may be chosen according to the extent of the area or areas within the esophagus to be treated. For example, a longer stent extending up to 25 or 30 cm may be used in a method of treatment or diagnosis to be performed on the entire length of the esophagus. Shorter stents may be used to treat a more localised area or areas.

The thickness of the stent coating will be dependent on factors such as the desired concentration of the active agent, the nature and amount of any other excipients which may be present in the coating, the presence of multiple coating layers (e.g. whether there is any base coat and/or top coat present), etc. Suitable thicknesses can readily be determined by those skilled in the art. Typically, these will be of the order of several micrometres, e.g. from about 3 to about 20 micrometres, or from about 5 to about 10 micrometres.

In cases where it is desirable that the stent should be biodegradable, the thickness of the stent body and coating should be selected accordingly. Suitable thicknesses may readily be selected such that the stent degrades entirely in vivo over a time period ranging from 2 to 24 hours.

Suitable active agents for use in the invention include known photosensitising agents or precursors of such agents. These includes, for example, psoralens, chlorins, phthalocyanins or porphyrins or protoporphyrin precursors (e.g. naturally occurring precursors) which are structural precursors of protoporphyrin and derivatives thereof, for example 5-ALA, porphobilinogen or precursors or derivatives (e.g. 5-ALA esters) thereof. Examples of active agents include, but are not limited to:
Hematoporphyrin derivative (HpD) (a complex mixture of porphyrins derived from hematoporphyrin);
Hematoporphyrines such as Photofrin® (Quadra Logic Technologies Inc., Vancouver, Canada) and Hematoporphyrin IX (HpIX);
Photosan III (Seehof Laboratorium GmbH, Seehof, Wesselburenerkoog, Germany);
Chlorins such as tetra (m-hydroxyphenyl) chlorins (m-THPC) and their bacteriochlorins (Scotia Pharmaceuticals Ltd, Surrey, UK), mono-L-aspartyl chlorin e6 (NPe6) (Nippon Petrochemical Co., Calif., USA), chlorin e6 (Porphyrin Products Inc.), benzoporphyrins (Quadra Logic Technologies Inc., Vancouver, Canada) (e.g. benzoporphyrin derivative monoacid ring A, BPD-MA) and purpurines (PDT Pharmaceuticals Inc., Calif., USA) (e.g. tin-ethyl etiopurpurin, SnET2);
Phthalocyanines (e.g. zinc-(Quadra Logic Technologies Inc., Vancouver, Canada), some aluminium- or silicon phthalocyanines, which may be sulphonated, in particular sulphonated phthalocyanines such as aluminium phthalocyanine di-sulphonate (AIPcS₂ₐ) or aluminium phthalocyanine tetra-sulphonate (AIPcS₄));
Porphycenes;
Hypocrellins;
Protoporphyrin IX (PpIX);
Hematoporphyrin di-ethers;
Uroporphyrins;
Coproporphyrins;
Deuteroporphyrin;
Polyhematoporphyrin (PHP);
Lutetium texaphyrin (Lu-Tex);
5-ALA and esters of 5-ALA;
Porphobilinogen;
and precursors and derivatives thereof.

In a preferred embodiment, the active agent is 5-ALA or a precursor or derivative thereof.

The term “5-ALA” denotes 5-aminolevulinic acid, i.e. 5-amino-4-oxo-pentanoic acid.

The term “precursor of 5-ALA” denotes compounds which are converted metabolically to 5-ALA and which are thus essentially equivalent to 5-ALA. Thus the term “precursor of 5-ALA” covers biological precursors for protoporphyrin in the metabolic pathway for haem biosynthesis.

The term “derivative of 5-ALA” denotes chemically modified 5-ALA, i.e. 5-ALA having undergone a chemical derivation such as substitution of a chemical group or addition of a further chemical group to modify or change any of its physico-chemical properties such as solubility or lipophilicity. Chemical derivation is preferably carried out at the carboxy group of 5-ALA, at the amino group of 5-ALA or at the keto group of 5-ALA, more preferably at the carboxy group or the amino group of 5-ALA. Preferred derivatives include esters, amides and ethers of 5-ALA, most preferred 5-ALA esters.

Preferred active agents for use in the invention are 5-ALA and pharmaceutically acceptable salts thereof, precursors of 5-ALA and pharmaceutically acceptable salts thereof, and derivatives of 5-ALA and pharmaceutically acceptable salts thereof (e.g. 5-ALA esters and their salts).

The term “pharmaceutically acceptable salt” denotes a salt that is suitable for use in a pharmaceutical product and which fulfils the requirements related to for instance safety, bioavailability and tolerability (see for instance P. H. Stahl et al. (eds.) Handbook of Pharmaceutical Salts, Publisher Helvetica Chimica Acta, Zurich, 2002).

The use of 5-ALA and 5-ALA esters both in methods of photodynamic therapy (PDT) and photodynamic diagnosis (PDD) is well known in the scientific and patent literature (see, for example, WO 2006/051269, WO 2005/092838, WO 03/011265, WO 02/09690, WO 02/10120, WO 2003/041673 and U.S. Pat. No. 6,034,267, the contents of which are incorporated herein by reference). All such 5-ALA esters and their pharmaceutically acceptable salts are suitable for use in the diagnostic methods herein described.

The 5-ALA esters for use in the invention may be any ester of 5-ALA capable of forming protoporphyrins, e.g. PpIX or a PpIX derivative (e.g. a PpIX ester) in vivo. Typically, such esters will be a precursor of PpIX or of a PpIX derivative in the biosynthetic pathway for haem and which are therefore capable of inducing an accumulation of PpIX following administration in vivo.

Esters of 5-ALA which are N-substituted may be used in the invention. However, those compounds in which the 5-amino group is unsubstituted are particularly preferred. Such compounds are generally known and described in the literature; see, for example, WO 96/28412 and WO 02/10120 to Photocure ASA, the contents of which are incorporated herein by reference.

Esters resulting from a reaction of 5-ALA with substituted or unsubstituted alkanols, i.e. alkyl esters and substituted alkyl esters, and pharmaceutically acceptable salts thereof, are especially preferred for use in the invention. Examples of such compounds include those of general formula I and pharmaceutically acceptable salts thereof:

R²₂N—CH₂COCH₂—CH₂CO—OR¹ (I)

wherein
R¹ represents an optionally substituted alkyl group; and
R² each independently represents a hydrogen atom or a group R¹.

As used herein, the term “alkyl”, unless stated otherwise, includes any long or short chain, cyclic, straight-chained or branched, saturated or unsaturated aliphatic hydrocarbon group. Unsaturated alkyl groups may be mono- or polyunsaturated and include both alkenyl and alkynyl groups. Unless stated otherwise, such alkyl groups may contain up to 40 carbon atoms. However, alkyl groups containing up to 30 carbon atoms, preferably up to 10, particularly preferably up to 8, especially preferably up to 6 carbon atoms, are preferred.

In the compounds of formula I, the R¹ groups are substituted or unsubstituted alkyl groups. If R¹ is a substituted alkyl group, one or more substituents are either attached to the alkyl group and/or interrupt the alkyl group. Suitable substituents that are attached to the alkyl group are those selected from hydroxy, alkoxy, acyloxy, alkoxycarbonyloxy, amino, aryl, nitro, oxo, fluoro, —SR³, —NR³₂ and —PR³₂, wherein R³ is a hydrogen atom or a C₁₋₆ alkyl group. Suitable substituents that interrupt the alkyl group are those selected from —O—, —S— or —PR³ (where R³ is as hereinbefore defined).

In one embodiment, R¹ may be an alkyl group substituted with one or more aryl substituents, i.e. aryl groups, preferably an alkyl group substituted with one aryl group.

As used herein, the term “aryl group” denotes an aromatic group which may or may not contain heteroatoms like nitrogen, oxygen or sulfur. Aryl groups which do not contain heteroatoms are preferred. Preferred aryl groups comprise up to 20 carbon atoms, more preferably up to 12 carbon atoms, for example, 10 or 6 carbon atoms.

Preferred embodiments of aryl groups are phenyl and naphthyl, especially phenyl. Further, the aryl group may optionally be substituted by one or more, more preferably one or two, substituents. Preferably, the aryl group is substituted at the meta- or para- position, most preferably the para-position. Suitable substituents include haloalkyl (e.g. trifluoromethyl), alkoxy (preferably alkoxy groups containing 1 to 6 carbon atoms), halo (e.g. iodo, bromo, chloro or fluoro, preferably chloro or fluoro), nitro and C₁₋₆ alkyl (preferably C₁₋₄ alkyl). Preferred C₁₋₆ alkyl groups include methyl, isopropyl and t-butyl, particularly methyl. Particularly preferred aryl substituents are chloro and nitro. However, still more preferably the aryl group is unsubstituted.

Preferred such aryl-substituted R¹ groups include benzyl, 4-isopropylbenzyl, 4-methylbenzyl, 2-methylbenzyl, 3-methylbenzyl, 4-[t-butyl]benzyl, 4-[trifluoromethyl]benzyl, 4-methoxybenzyl, 3,4-[di-chloro]benzyl, 4-chlorobenzyl, 4-fluorobenzyl, 2-fluorobenzyl, 3-fluorobenzyl, 2,3,4,5,6-pentafluorobenzyl, 3-nitrobenzyl, 4-nitrobenzyl, 2-phenylethyl, 4-phenylbutyl, 3-pyridinyl-methyl, 4-diphenyl-methyl and benzyl-5-[(1-acetyloxyethoxy)-carbonyl]. More preferred such R¹ groups are benzyl, 4-isopropylbenzyl, 4-methylbenzyl, 4-nitrobenzyl and 4-chlorobenzyl. Most preferred is benzyl.

If R¹ is a substituted alkyl group, one or more oxo substituents are preferred. Preferably, such groups are straight-chained C₄₋₁₂ alkyl groups which are substituted by one or more oxo groups, preferably by one to five oxo groups. The oxo groups are preferably present in the substituted alkyl group in an alternating order, i.e. resulting in short chain polyethylene glycol substituents. Preferred examples of such groups include 3,6-dioxa-1-octyl and 3,6,9-trioxa-1-decyl. In another preferred embodiment, R¹ is an alkyl group interrupted by one or more oxygen atoms (ether or polyether group), preferably a straight-chained C₄₋₁₂ alkyl and more preferably a straight-chained C₆₋₁₀ alkyl group being interrupted by 1 to 4 oxygen atoms, more preferably a straight-chained polyethylene glycol group (—(CH₂)₂—O—), with n being an integer of from 1 to 5.

If R¹ is an unsubstituted alkyl group, R¹ groups that are saturated straight-chained or branched alkyl groups are preferred. If R¹ is a saturated straight-chained alkyl group, C₁₋₁₀ straight-chained alkyl groups are preferred. Representative examples of suitable straight-chained alkyl groups include methyl, ethyl, n-propyl, n-butyl, n-pentyl, n-hexyl and n-octyl. Particularly preferred are C₁₋₆ straight-chained alkyl groups. Most particularly preferred is n-hexyl. If R¹ is a saturated branched alkyl group, such branched alkyl groups preferably consist of a stem of 4 to 8, preferably 5 to 8 straight-chained carbon atoms and said stem is branched by one or more C₁₋₆ alkyl groups, preferably C₁₋₂ alkyl groups. Examples of such saturated branched alkyl groups include 2-methylpentyl, 4-methylpentyl, 1-ethylbutyl and 3,3-dimethyl-1-butyl.

In the compounds of formula I, each R² independently represents a hydrogen atom or a group R¹. Particularly preferred for use in the invention are those compounds of formula I in which at least one R² represents a hydrogen atom. In especially preferred compounds each R² represents a hydrogen atom.

Preferred 5-ALA esters for use in the invention include methyl ALA ester, ethyl ALA ester, propyl ALA ester, butyl ALA ester, pentyl ALA ester, hexyl ALA ester, octyl ALA ester, 2-methoxyethyl ALA ester, 2-methylpentyl ALA ester, 4-methylpentyl ALA ester, 1-ethylbutyl ALA ester, 3,3 -dimethyl-1-butyl ALA ester, benzyl ALA ester, 4-isopropylbenzyl ALA ester, 4-methylbenzyl ALA ester, 2-methylbenzyl ALA ester, 3-methylbenzyl ALA ester, 4-[t-butyl]benzyl ALA ester, 4-[trifluoromethyl]benzyl ALA ester, 4-methoxybenzyl ALA ester, 3,4-[di-chlorobenzyl ALA ester, 4-chlorobenzyl ALA ester, 4-fluorobenzyl ALA ester, 2-fluorobenzyl ALA ester, 3-fluorobenzyl ALA ester, 2,3,4,5,6-pentafluorobenzyl ALA ester, 3-nitrobenzyl ALA ester, 4-nitrobenzyl ALA ester, 2-phenylethyl ALA ester, 4-phenylbutyl ALA ester, 3 -pyridinyl-methyl ALA ester, 4-diphenyl-methyl ALA ester and benzyl-5-[(1-acetyloxyethoxy)-carbonyl]amino levulinate, and 3-methylbenzyl ALA ester.

The 5-ALA esters for use in the invention may be in the form of a free amine, e.g. —NH₂, —NHR² or —NR²R² or preferably in the form of a pharmaceutically acceptable salt. Such salts preferably are acid addition salts with pharmaceutically acceptable organic or inorganic acids. Suitable acids include, for example, hydrochloric, nitric, hydrobromic, phosphoric, sulfuric, sulfonic and sulfonic acid derivatives (the salts of ALA-esters and the latter acids are described in WO 2005/092838 to Photocure ASA, the entire contents of which are incorporated herein by reference). A preferred acid is hydrochloride acid, HCl. Further preferred acids are nitric acid, sulfonic acid and sulfonic acid derivatives (e.g. mesylate or tosylate). Procedures for salt formation are conventional in the art and are for instance described in WO 2005/092838.

Preferred compounds of formula I and pharmaceutically acceptable salts thereof for use in the invention are those wherein R¹ represents an unsubstituted alkyl group, preferably an unsubstituted, saturated, straight-chained or branched, alkyl group (e.g. a C₁₋₁₀ alkyl group). Particularly preferred compounds are those wherein R¹ is hexyl, more preferably n-hexyl, and both R² represent hydrogen, i.e. 5-ALA hexyl ester and pharmaceutically acceptable salts thereof. The preferred compound for use in the invention is 5-ALA hexyl ester and its pharmaceutically acceptable salts, preferably the hydrochloride salt, nitrate salt or sulfonic acid salts or sulfonic acid derivative salts, such as mesylate, tosylate or napsylate.

5-ALA esters and pharmaceutically acceptable salts thereof for use in the invention may be prepared by any conventional procedure available in the art, e.g. as described in WO 96/28412, WO 02/10120, WO 03/041673 and in N. Fotinos et al., Photochemistry and Photobiology 2006: 82: 994-1015 and the cited literature references therein. Briefly, 5-ALA esters may be prepared by reaction of 5-ALA with the appropriate alcohol in the presence of a catalyst, e.g. an acid. Pharmaceutically acceptable salts of 5-ALA esters may be prepared as described hereinbefore by reaction of a pharmaceutically acceptable 5-ALA salt, e.g. 5-ALA hydrochloride with the appropriate alcohol. Alternatively compounds for use in the invention like 5-ALA hexyl ester may be available commercially, e.g. from Photocure ASA, Norway.

Suitable coating materials and methods for application of these to the stent body, in particular to its tissue-contacting surface, are generally known in the art, e.g. in PCT/EP2011/061688 to Photocure ASA, the contents of which are hereby incorporated by reference. These should be capable of securing an effective amount of the desired active agent onto the body of the stent (the active should be present in a therapeutically or diagnostically effective amount); and able to keep this in place during delivery and expansion of the stent in vivo. In certain cases, the coating may be selected such that this controls the rate of delivery of the active agent from the stent to the esophageal wall.

In a further aspect the invention provides a method for preparing a stent as herein described, said method comprising the following steps:
(a) providing a generally cylindrical stent body; and
(b) applying to said body a coating comprising a photosensitising agent or a precursor thereof.

In one embodiment, the coating may be provided in the form of a powder, i.e. a dry, bulk solid composed of a large number of very fine particles, more preferably in the form of a compressed powder, i.e. having lost its ability to flow. In another embodiment, the dry pharmaceutical composition is in the form of a cake, i.e. a dry bulk solid formed into a small block. In a preferred embodiment, the coating is in the form of a film, i.e. one or more (thin) layers of dry/dried material, preferably a relatively homogeneous film which covers substantially the whole surface of the stent or substantially the outer surface of the stent. In a further preferred embodiment, this film is well attached and stays well attached to the stent surface, i.e. is relatively stable under mechanical stress which may occur during transport and shipment and during deployment in vivo.

In one embodiment, the coating may be obtained as a film by film coating processes known in the art, preferably by dip-coating or spray-coating.

Dip-coating techniques can be described as a process where the stent is immersed in a liquid and then withdrawn with a well-defined withdrawal speed under controlled temperature and atmospheric conditions. The coating thickness is mainly defined by the withdrawal speed, by the solid content and the viscosity of the liquid. In a dip-coating process, the stent body is immersed in a liquid, i.e. a solution or dispersion of the active ingredient and optionally one or more polymers and/or other pharmaceutically acceptable excipients in one or more suitable solvents. The stent is withdrawn from the liquid whereby the liquid is deposited. Withdrawal is preferably carried out at a constant speed to achieve a uniform coating. Concomitantly, excess liquid is drained from the surface of the stent. The solvent evaporates from the liquid, forming a film. This process can be accelerated by providing heat. For volatile solvents such as lower alcohols, evaporation starts during the deposition and drainage steps.

Spray-coating involves atomizing or aerosolizing of the liquid, i.e. a solution or dispersion of the active ingredient and optionally one or more polymers and/or optionally other pharmaceutically acceptable excipients in one or more suitable solvents, e.g. using a spray gun. Preferably, the spray gun can be adjusted horizontally, vertically and angularly and it can be swiveled too. Heaters can be used to accelerate evaporation of the solvent(s).

In another embodiment, the stent may be coated by solvent evaporation involving the use of a solvent in which the active agent is dissolved or dispersed. A liquid is prepared by dissolving or suspending the active agent and optionally one or more polymers and/or optionally other pharmaceutically acceptable excipients in one or more suitable solvents. Suitable solvents should be chosen such that these do not alter the activity of the active agent. Such solvents may readily be determined by those skilled in the art and include, for example, solvents with a low boiling point like for instance lower alcohols, ethers etc. The resulting liquid may be applied to the stent using known techniques such as immersion (e.g. dipping), brushing, spraying, etc. Spraying is particularly preferred since this provides a more uniform coating. Subsequent evaporation of the solvent(s) leaves a coating containing the active agent on the stent body. If necessary, the stent body may be exposed to heat for a period which is long enough to achieve complete evaporation of the solvent(s). During that period, the stent may be moved, e.g. rotated on a mandrel to promote evaporation and to ensure a homogeneous distribution of the liquid. Depending on the parameters of the evaporation like temperature and humidity, and the amount of liquid applied to the stent, the coating is obtained as a film or cake. These coating steps may, if desired, be repeated if necessary to achieve the desired effective amount of active ingredient on the stent body. Greater control over the amount of active agent may be achieved through the use of multiple thin coats.

The presence of a polymer in the final coating allows the active agent to be contained in a coating which is generally more resilient and which serves to retain the coating in place during expansion of the stent. Depending on the choice of polymer material for the coating, this may also serve to control the rate of release of the active agent following delivery to the esophageal surface. Release rates may further be tuned depending on the ratio of active agent to polymer in the coating (or, where present, multiple coatings).

In another embodiment, the stent coating is obtained by lyophilization. Briefly, in general the lyophilization process consists of three stages: freezing, primary drying and secondary drying. A liquid, i.e. solution or suspension of the compound(s) to be lyophilized is first frozen. Usually freezing temperatures are between −50 to −80° C., depending on which solvent(s) is used. During the primary drying phase, the pressure is lowered and enough heat is supplied to the frozen liquid for the solvent, usually water, to sublimate. In the secondary drying phase, unfrozen solvent molecules are removed. In order to obtain the coating by lyophilization, a liquid is prepared by dissolving or suspending the active ingredient and optionally one or more polymers and/or and optionally other pharmaceutically acceptable excipients in a suitable solvent, usually water. However, it is also possible to use mixtures of solvents, e.g. water and alcohols such as ethanol. The so-obtained liquid is applied to the stent. The stent may be cooled and/or moved, e.g. rotated, during the application of the liquid. The stent is frozen, for instance quickly frozen to avoid the formation of larger crystals. The stent is then lyophilized as described before. The coating is typically obtained in the form of a cake, a powder (which can be further compressed) or preferably a film.

Specific equipment for dip-coating, spray-coating and lyophilization is commercially available.

In a preferred embodiment, one or more polymers and optionally other pharmaceutically acceptable excipients are present in the coatings herein described. Preferred one or more polymers are polymers which have good film-forming properties and/or good gel forming properties. Preferred other pharmaceutically acceptable excipients are selected from one or more of the following compounds: plasticizers, coloring agents and thickening agents. Other pharmaceutically acceptable excipients which may be present in the coating are disintegrants, mucoadhesive agents, surface penetration enhancing agents and chelating agents.

If one or more polymers and/or pharmaceutically acceptable excipients are present in the coating, the active agent may be present in the range of 0.25 to 50%, for example 0.5 to 30%, such as 0.5 to 15% or 1 to 10% or 1 to 7% by weight of the total weight of the coating. Alternatively, if only one or more polymers are additionally present in the dry pharmaceutical composition, the active agent may be present in the range of 50 to 99%, for example 60 to 91% or 75 to 90% by weight of the total weight of the coating. By having a high amount of active agent compared to the amount of the one or more polymer, the liquid which is used to deposit the composition is less viscous and thus easier to handle and process. In another embodiment if one or more polymers and pharmaceutically acceptable excipients selected from plasticizers are present in the coating, the active agent may be present in the range of 15 to 85%, for example 20 to 80% such as 25 to 78% or 26 to 60% by weight of the total weight of the coating.

All one or more polymers and pharmaceutically acceptable excipients should be non-toxic, non-irritant and devoid of leachable impurities. They should be inert towards the active agent, i.e. should not promote its degradation. One or more of each pharmaceutically acceptable excipient compound may be used, e.g. one or more plasticizers, one or more coloring agents, etc.

The one or more polymers for use in the coatings can be natural, semi-natural, i.e. derivatives of natural polymers which are obtained by a chemical reaction, or synthetic polymers; they may be homopolymers or copolymers.

Preferably, polymers are used which have good film-forming properties, i.e. which Form—together with the active agent—a film when deposited on the stent surface. A preferred group of such polymers are starch, cellulose and derivatives of starch and cellulose. Preferred starch derivatives are starch acetate and carboxymethyl starches, preferably with an amylose content of at least 18% by weight. One preferred cellulose is microcrystalline cellulose. Other preferred cellulose derivatives are cellulose ethers such as methylcellulose, ethylcellulose, hydroxyethylcellulose, hydroxypropylcellulose, hydroxypropylmethylcellulose, hydroxypropylethyl-cellulose and carboxymethylcellulose. Such polymers may be used in combination with other polymers, e.g. ethylcellulose with hydroxypropylmethylcellulose. Other preferred cellulose derivatives are cellulose acetate phthalate and nitrocellulose. Further preferred polymers are rosin and rosin esters. Another preferred group of polymers are (meth) acrylate polymers and copolymers. The use of “meth” as a prefix in parenthesis indicates, in accordance with common practice, that the polymer molecule is derived from monomers having the carbon atom skeleton of either or both of acrylic acid and methacrylic acid. Such polymers and copolymers are e.g. based on methylmethacrylate, ethylacrylate, methacrylic acid and trimethylammonioethylmethacrylate chloride, e.g. anionic and cationic polymers of methacrylic acid, copolymers of methacrylates, copolymers of acrylates and methacrylates, copolymers of ethylacrylates and methylmethacrylates. Other preferred polymers are polyvinyl acetate phthalate. In a more preferred embodiment, cellulose and cellulose derivatives, especially cellulose ethers, are used as one or more polymers in the coatings herein described.

In another embodiment, polymers with good gel-forming properties may be used, i.e. which form—together with the active agent—gels on contact with water and fluids of mucosa lined surfaces. Preferred such polymers are gums, preferably gellan gum, xanthan gum and carrageenan. Other preferred polymers are chitin, chitosan and chitosan derivatives such as chitosan salts (hydrochloride, lactate, aspartate, glutamate) and N-acetylated chitosan or N-alkylated chitosan. Yet other preferred polymers are pectin, alginates, e.g. sodium alginate, pullulan, hyaluronic acid and derivatives thereof.

Preferably, in yet another embodiment, polymers with good film-forming properties and good gel-forming properties are used, e.g. cellulose ethers like methylcellulose, ethylcellulose, gellan gum, chitosan and chitosan derivatives, pullulan, alginates, hyaluronic acid, derivatives of hyaluronic acid or carrageenan. Preferred such polymers are cellulose ethers like methylcellulose, ethylcellulose, hydroxyethylcellulose, hydroxypropylcellulose, hydroxypropylmethylcellulose, hydroxypropylethylcellulose and carboxymethylcellulose and chitosan and chitosan derivatives.

Polymers with good film-forming properties are preferably used if the coating should form a film.

The polymers may be water soluble or insoluble in water. In a preferred embodiment, water soluble polymers are used.

If present in the coating, the one or more polymers may conveniently be provided in a concentration range of 50 to 99.75%, for example 70 to 99.5%, e.g. 85 to 99.5%, or 90 to 99% or 93 to 99% by weight of the total weight of the coating. Alternatively, if only one or more polymers are additionally present in the coating, the one or more polymers may be present in the range of 1 to 50%, for example 9 to 40% or 10 to 25% by weight of the total weight of the coating. By having a high amount of active agent compared to the amount of the one or more polymer, the liquid which is used to deposit the composition is less viscous and thus easier to handle and process. In another embodiment if one or more polymers and pharmaceutically acceptable excipients selected from plasticizers are present in the coating, the one or more polymers may be present in the range of 20 to 65%, for example 25 to 62% such as 30 to 55% or 40 to 54% by weight of the total weight of the coating.

Other pharmaceutically acceptable excipients which may be present in the coating are plasticizers. In general their use is to reduce the glass transition temperature of a polymer making it more elastic and deformable, i.e. flexible. Hence they may be present in the coating if one or more polymers are present, preferably if one or more film-forming polymers are present. Plasticizers are preferably chosen in such a way that they work well with the given polymer(s). In one embodiment, suitable plasticizers are acting as a good solvent for the polymer(s) in question. In another embodiment, if water soluble polymers are used, the plasticizer is preferably a water miscible compound. Suitable plasticizers are low molecular weight polyethylene glycols, phthalate derivatives like dimethyl, diethyl and dibutyl phthalate, citrate esters such as triethyl, tributyl and acetyl citrate, dibutyl sebacate, camphor, triacetin, oils and glycerides such as castor oil, acetylated monoglycerides and fractionated coconut oil. Glycerol and propylene glycol are also common plasticizers, they should however not be used if the coating contains as an active ingredient lower alkyl ALA esters or salts thereof, such as C₁-C₈-alkyl ALA esters since they may promote degradation of such active ingredients.

If present in the coating, the plasticizers may conveniently be provided in a concentration range of 1 to 30%, for example 5 to 20% or 7 to 15% by weight of the total polymer weight. Alternatively, the plasticizers may be provided in a higher concentration range, for instance in a concentration range of 10 to 175%, or 35 to 150% or 37 to 80% by weight of the total polymer weight.

Other pharmaceutically acceptable excipients which may be present in the coating are coloring agents, such as synthetic dyes or pigments, e.g. titanium dioxide or yellow iron oxide. Pigments usually decrease the permeability of the coating to water vapor and oxygen and may thus increase its shelf life. Further, they contribute to the total solids of the liquid used to obtain the coating without significantly contributing to its viscosity. Thus faster processing time by virtue of more rapid drying is possible, which is particularly significant for aqueous based liquids used in spray-coating. If present in the coating, the coloring agents may conveniently be provided in a concentration range of 0.1 to 20%, for example 0.5 to 10% or 1 to 5% by weight of the coating.

Other pharmaceutically acceptable excipients which may be present in the coating are thickening agents. Such agents swell as they absorb liquid and thus may be used to improve the viscosity and consistency of the liquid which is used to obtain the coating. Preferably, thickening agents are used in liquids which are employed in dip-coating. The choice of thickening agents is dependent on whether the liquid is an aqueous or aqueous based liquid or whether non-aqueous solvents are used to form the liquid. Some of the afore-mentioned polymers have thickening properties, e.g. gums like guar gum, cellulose derivatives like carboxymethylcellulose and (meth)acrylates. Other suitable thickening agents are polyacrylic acids (carbomer) or wax or a waxy solids e.g. solid fatty alcohols or solid fatty acids. If present in the coating, the thickening agents may conveniently be provided in such an amount that the desired viscosity of the liquid described above is obtained. The actual amount will depend on the one or more solvent said liquid comprises and the nature of the thickening agent.

Other pharmaceutically acceptable excipients which may be present in the coating are disintegrants. Generally, disintegrants aid in the break up of the coating when it is put into a moist environment. Some of the aforementioned polymers do exhibit disintegrant properties, e.g. certain celluloses, starch and derivatives thereof. If these polymers are present, there may not be a need or desire to add any further disintegrants. More effective disintegrants are referred to as superdisintegrants. Those include for instance alginic acid, croscarmellose, crospovidone and sodium starch glycolate. Such compounds swell when they come in contact with fluids but they do not form a gel which would decrease their disintegration properties. If present in the coating, the disintegrants may conveniently be provided in a concentration range of 0.1 to 10% by weight of the total weight of the coating, for example 0.25 to 5% or 0.5 to 4% by weight.

The coatings may further comprise one or more mucoadhesive agents. The term “mucoadhesive agent” denotes a compound which exhibits an affinity for a mucosa surface, i.e. which adheres to that surface through the formation of bonds which are generally non-covalent in nature, whether binding occurs through interaction with the mucous and/or the underlying cells.

Suitable mucoadhesive agents may be natural or synthetic compounds, polyanionic, polycationic or neutral, water-soluble or water-insoluble, but are preferably large, e.g. having a molecular weight of 500 kDa to 3000 kDa, e.g. 1000 kDa to 2000 kDa, water-insoluble cross-linked, e.g. containing 0.05% to 2% cross-linker by weight of the total polymer, prior to any hydration, water-swellable polymers capable of forming hydrogen bonds. Preferably such mucoadhesive compounds have a mucoadhesive force greater than 100, especially preferably greater than 120, particularly greater than 150, expressed as a percent relative to a standard in vitro, as assessed according to the method of Smart et al., 1984, J. Pharm. Pharmacol., 36, pp 295-299.

Some of the afore-mentioned polymers exhibit mucoadhesive properties, for instance gums like guar gum, chitosan and chitosan derivatives, pullulan, sodium alginate or hyaluronic acid. If these polymers are present, there may not be a need or desire to add any further mucoadhesive agents.

Suitable mucoadhesive agents are selected from polysaccharides, preferably dextran, pectin, amylopectin or agar; gums, preferably guar gum or locust bean gum; salts of alginic acid, e.g. magnesium alginate; poly(acrylic acid) and crosslinked or non-crosslinked copolymers of poly(acrylic acid) and derivatives of poly(acrylic acid) such as salts and esters like for instance carbomer (carbopol).

When present, the mucoadhesive agent may conveniently be provided in a concentration range of 0.05 to 30%, e.g. about 1 to 25% by weight of the total weight of the coating.

The coating may further comprise one or more surface penetration enhancing agents. Such agents may have a beneficial effect in enhancing the photosensitizing effect of the active agent, e.g. of 5-ALA, the derivative of 5-ALA or the precursor of 5-ALA present in the coating. Preferred surface penetration enhancing agents are chelators (e.g. EDTA), surfactants (e.g. sodium dodecyl sulfate), non-surfactants, bile salts (sodium deoxycholate), fatty alcohols e.g. oleylalcohol, fatty acids, e.g. oleic acid and esters of fatty acids and alcohol, e.g.

isopropylmyristate. When present, the surface penetration enhancing agent may conveniently be provided in a concentration range of 0.2 to 20% by weight of the total weight of the coating, e.g. about 1 to 15% or 0.5 to 10% by weight of the total weight of the coating.

The coating may further comprise one or more chelating agents. Such agents may also have a beneficial effect in enhancing the photosensitizing effect of the active agent, e.g. of 5-ALA, the derivative of 5-ALA or the precursor of 5-ALA present in the coating. Their ability to enhance the photosensitizing effect of the active agent means that a lower amount of the active agent needs to be taken up by the cells in order to achieve the desired photodynamic effect. This has the advantage that less time is generally needed for the cells to take up an effective amount of the photosensitizing agent or precursor thereof.

Chelating agents may, for example, be included in order to enhance the accumulation of PpIX since the chelation of iron by the chelating agent prhomogeneousts its incorporation into PpIX to form haem by the action of the enzyme ferrochelatase, thereby leading to a build up of PpIX. The photosensitizing effect is therefore enhanced. Suitable chelating agents are aminopolycarboxylic acids and any of the chelants described in the literature for metal detoxification or for the chelation of paramagnetic metal ions in magnetic resonance imaging contrast agents. Particular mention may be made of EDTA, CDTA (cyclohexane triamine tetraacetic acid), DTPA and DOTA and well known derivatives and analogues thereof. EDTA and DTPA are particularly preferred. Other suitable chelating agents are desferrioxamine and siderophores and they may be used alone or in conjunction with aminopolycarboxylic acid chelating agents such as EDTA. Some of these chelating agents do also exhibit surface penetration assisting agent properties, e.g. EDTA. Preferred chelators are iron-chelators such as CP 94 (1,2-diethyl-3-hydroxypyridin-4-one) as described in Bech et al., Journal of Photochemistry and Photobiology B: Biology 41 (1997), 136-144, or Deferasirox such as described in US-A-6,596,750. Where present, the chelating agent may conveniently be used at a concentration of 0.01 to 12%, e.g. 0.1 to 5% by weight based on the total weight of the coating.

The coating may further comprise one or more pharmaceutically acceptable excipients which are different from the afore-mentioned excipients. Such excipients are for instance surfactants, emulsifiers, preferably non-ionic or cationic emulsifiers, fillers, binders, spreading agents, stabilizing agents or preservatives. The skilled man will be able to select suitable excipients based on their purpose. Common excipients that may be used in the pharmaceutical products herein described are listed in various handbooks (e.g. D. E. Bugay and W. P. Findlay (Eds) Pharmaceutical excipients (Marcel Dekker, New York, 1999), E- M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe fur Pharmazie, Kosmetik and angrenzende Gebiete (Edition Cantor Aulendorf, 1989)).

All of the above-mentioned pharmaceutically acceptable excipients are well known in the art and are commercially available from various manufacturers.

Following preparation, the coated stent should be covered or sealed such that this is retained in a moisture-free environment prior to use, for example in an airtight/moisture tight bag.

The stent according to the invention can be used to deliver the active agent to the esophageal wall by introducing the stent down the esophagus and, if necessary, radially expanding this into contact with the desired portion of the esophageal wall. Esophageal delivery may be achieved using known techniques such as by a catheter or using other stent expanding devices. Radial expansion may be achieved by balloon expansion of the stent (e.g. where a balloon catheter is used) or by self-expansion. When the stent is balloon-expandable, this will generally be formed in the expanded state, crimped onto a balloon dilation catheter or other stent expanding device for delivery. At the desired site within the esophagus, the stent is expanded into place, e.g. by the radial expansion of the balloon

The active agent is released from the stent coating in a moist environment, i.e. upon contact with the mucosa-lined surface of the esophagus. In order to achieve a full release of the active agent, the one or more polymers or other pharmaceutically acceptable excipients need to be chosen in such a way that they are either dissolved upon contact with the mucosa-lined surfaces of the esophagus or at least disintegrate to allow release of the active ingredient. The excipients, especially any polymers present, thus need to be dissolved or disintegrated at the given pH on said mucosa-lined surface.

In a further aspect the invention thus provides an esophageal stent having a coating capable of releasing an active agent which is a photosensitizing agent or a precursor thereof. Preferably, the active agent is a 5-ALA ester or a pharmaceutically acceptable salt thereof, e.g. 5-ALA hexyl ester.

The release profile (immediate/quick, sustained and delayed release) and the residence time of the coating at the target area of the esophagus in which treatment or diagnosis is to be performed, can be influenced by the choice of the polymer as well. A quick release of the active agent may be preferred if a comparably high concentration of the active agent at the site of treatment is desired. A delayed release of the active ingredient may be preferred if a low concentration of the active agent at the site of treatment is desired. For both PDD and PDT, an immediate release of the active is generally preferred, however, for PDT a longer release time may be acceptable in cases where this leads to an effective concentration of photosensitizer. Preferred are coatings formulated for release of the active ingredient within a period of from 15 minutes to 1 hour, preferably 20 to 45 minutes, following deployment of the stent.

Following delivery of the active agent to the target site, the stent may be removed prior to carrying out PDD and/or PDT. Depending on the nature of the stent body, however, this may not be necessary and indeed, in some cases, it can be desirable to leave the stent in situ whilst carrying out photoactivation since this has the benefit of holding open the esophagus such that the light dose necessary to activate the photosensitizer can readily reach the esophageal surface. Polymeric stents, for example, may be transparent to light or at least sufficiently transparent that they permit an effective light dose to reach the target site and, in the case of PDD, to permit the resulting fluorescence to be observed. Such “transparent” stents therefore need not be removed before PDD or PDT. After PDD or PDT has been carried out these may either be removed or, in the case where these are biodegradable, may be left at the site. Metallic stents are not transparent to light and so will typically be removed before carrying out photoactivation.

Once the active agent has been delivered and the photosensitizing agent has reached an effective tissue concentration at the desired site in the esophagaus, the site to be treated is then exposed to light to achieve the desired photoactivation and photodynamic treatment or diagnosis. The length of time between administration (i.e. delivery of the stent in vivo) and exposure to light will depend on the nature of the active agent and the nature of the coating. Generally, it is necessary that the active agent within the coating is sufficiently released to be taken up by the cells of the target tissue, if necessary converted into a photosensitiser, and achieves an effective tissue concentration at the site prior to photoactivation. The incubation time may be up to about 10 hours, e.g. about 30 minutes to 10 hours, preferably 1 hour to 7 hours, e.g. 2 hours to 5 hours.

The irradiation may be applied for a short time with a high light intensity, i.e. a high fluence rate or for a longer time with a low light intensity, i.e. low fluence rate. Generally, a higher light intensity will be used such that irradiation can be applied for a shorter time.

The wavelength of light used for irradiation may be selected to achieve an efficacious photodynamic effect. Light having wavelengths of between 300-800 nm, for example, the range 400-700 nm has been found to be particularly effective. For PDT, it can be particularly important to include the wavelengths 630 and 690 nm. Red light (600-670 nm) is particularly preferred since light at this wavelength is known to penetrate well into tissue. For PDD, blue light having a wavelength typically ranging from 380 to 450 nm will generally be used, giving rise to fluorescence which is measured in the red region (e.g. 550 to 750 nm). For both PDT and PDD, a single irradiation may be used or alternatively light may be split and delivered in a number of fractions, e.g. a few to several minutes between irradiations. Multiple irradiations may also be applied but are not preferred.

Methods for irradiation, e.g. by lamps or lasers, are well known in the art. The irradiation will in general be applied at a dose level of 10 to 100 Joules/cm² with an intensity of 20-200 mW/cm² when a laser is used, or a dose of 10-100 J/cm² with an intensity of 50-150 mW/cm² when a lamp is applied. Conveniently, a fibre optic carrying a laser light may be fed through an endoscope to accomplish exposure to irradiation. Exposure may be adjusted, as required, by varying the time of exposure and/or the light intensity.

In a preferred embodiment, the stent according to the invention may be used in a method of photodynamic diagnosis of an abnormality of the epithelial lining of the esophagus.

As used herein, the term “diagnosis” encompasses not only the detection (i.e. identification) of an abnormality, but also monitoring or surveillance of its progression and development. Abnormalities of the epithelial lining of the esophagus encompass not only cancerous conditions but also conditions which may be considered pre-cancerous or non-cancerous.

The term “pre-cancerous condition” denotes a tissue abnormality which, if left untreated, may ultimately lead to cancer. It is a generalized state associated with a significantly increased risk of cancer. A pre-cancerous condition may manifest itself by extensive and/or abnormal proliferation of cells, e.g. dysplasia and/or neoplasia.

Cancerous conditions in the esophagus include basal cell carcinomas and adenocarcinomas.

The term “non-cancerous conditions” includes tissue abnormalities with no or low malignant potential such as metaplasia.

In a preferred embodiment, the stent may be used in the treatment or diagnosis of Barrett's esophagus. As used herein, the term “Barrett's esophagus” is intended to encompass the presence of metaplastic epithelium. In later stage Barrett's esophagus, the presence of dysplastic tissue may be observed.

The term “metaplastic” denotes cells or tissues which have been reversibly replaced by another differentiated cell type. Metaplastic tissue is considered non-carcinogenic. The term “dysplastic” denotes the abnormal development or growth of cells or tissues. The term “neoplastic” denotes the abnormal proliferation of cells or tissues. Both the presence of dysplastic and neoplastic tissues is considered a pre-cancerous condition.

In another embodiment, the stent may be deployed in a method of photodynamic diagnosis of early stage Barrett's esophagus, i.e. detection of the presence of metaplastic cells. Once metaplasia has been detected, this may also be used in photodynamic methods for treatment of the condition and/or in photodynamic methods of monitoring the progression of the disease, for example in detecting early and/or late stage dysplasia. Monitoring of patients with Barrett's esophagus at regular intervals, e.g. annually or bi-annually, enables early detection of any progression of the disease to pre-cancerous (e.g. dysplastic and neoplastic) and/or cancerous (e.g. adenocarcinoma) stages. As will be appreciated, early identification of areas of the esophagus which are at high-risk of developing into cancer improves the chances of successful treatment, e.g. by operative intervention.

In another aspect the invention thus provides a stent as herein described for use in the photodynamic therapy or photodynamic diagnosis of an abnormality of the esophagus. In one embodiment, the abnormality is Barrett's esophagus. In another embodiment, the abnormality is esophageal cancer (e.g. esophageal adenocarcinoma).

When carrying out photodynamic diagnosis, fluorescence from the excited photosensitizer may be detected using any conventional fluorescence detector. The emitted fluorescence (e.g. at about 635 nm) is used to selectively detect abnormal cells or tissues, e.g. affected cancerous tissue or pre-cancerous lesions (dysplasia) or non-cancerous conditions (e.g. metaplasia). In a preferred embodiment of the invention, the fluorescence detected may be used to produce an image of the desired area or areas of the esophagus. Different levels of fluorescence intensity may then be used to identify the different types of cells which may be present. Those areas in which pre-cancerous (e.g. dysplastic) or cancerous cells are present give rise to a higher fluorescence intensity, i.e. a positive fluorescence contrast, compared to normal squamous epithelial tissue. Those areas in which metaplastic cells are present give rise to a lower fluorescence intensity, i.e. a negative fluorescence contrast, compared to normal cells. Thus, positive and negative contrast in a fluorescence intensity image may be used to map the different areas of the esophagus.

In a further aspect the invention thus provides a method of in vivo diagnosis of a portion of the epithelial surface of the esophagus, said method comprising the following steps:
(a) delivering to the esophagus of a subject, e.g. a human or non-human animal, an esophageal stent as herein described;
(b) waiting for a time period necessary for the photosensitising agent to achieve an effective tissue concentration at the desired site in the esophagus;
(c) optionally removing the stent;
(d) photoactivating the photosensitising agent;
(e) detecting fluorescence from said photosensitising agent using a fluorescence detector; and
(f) optionally converting the detected fluorescence into an image of an area of interest in the surface of the esophagus.

Detection of fluorescence may be used to identify the presence of certain abnormal cell or tissue types, for example, any of metaplastic, dysplastic or neoplastic cells or tissues. Alternatively, measurement of fluorescence intensity from the esophagus may be used to distinguish between the different types of cells or tissues which may be present. Identification of any high-risk areas (e.g. those comprising dysplastic or neoplastic cells) for operative intervention forms a preferred aspect of the invention.

The results from any of the diagnostic methods which are described herein may also provide a useful guide for any subsequent biopsy and/or as a guide to the areas for subsequent treatment. In one embodiment, the methods of diagnosis may therefore be performed prior to carrying out a biopsy and/or therapeutic treatment.

Suitable endoscopes for use in carrying out the methods herein described are known in the art and may be adapted to allow emission of blue light (for excitation purposes) in addition to white light, e.g. by being equipped with an internal filter assembly which passes primarily blue light. A foot pedal may be used to allow convenient switching between white and blue light. The light source may be a laser or a lamp. To visualize fluorescence, the endoscope may be equipped with an integrated filter which blocks most of the reflected blue light. A camera, such as a modified color charge-coupled device (CCD) camera, may be used to capture images of the esophagus and a standard color monitor may be used to display images of the esophagus. Irradiation is preferably performed for 5 to 30 minutes. A single irradiation may be used or alternatively a light split dose in which the light dose is delivered in a number of fractions, e.g. a few minutes to a few hours between irradiations, may be used. Multiple irradiations may also be applied. The area of examination may further be inspected by use of white light, e.g. before, during or after irradiation with blue light. Cancerous tissue or pre-cancerous lesions identified due to its fluorescence may be removed during irradiation or in white light.

Following identification of the abnormality using any of the diagnostic methods herein described, this may then be treated through alternative therapeutic techniques, e.g. by surgical or chemical treatment. Examples of current treatments include surgical treatment, endoscopic ablation therapy, chemical ablation (e.g. by

PDT), thermal ablation or mechanical ablation. In one embodiment of the invention, further application of the active agent (e.g. a 5-ALA ester compound) at the site of interest may be carried out in order to effect PDT. It will be appreciated that PDT may require higher concentrations of 5-ALA esters for destruction of the abnormal cells or tissues than used in the diagnostic methods. Generally, concentration ranges of up to 50% by weight, preferably 10 to 50% by weight, e.g. 15 to 30% by weight, are suitable.

In a further aspect the invention thus provides a method of treating an abnormality of the epithelial-lined surface of the esophagus, said method comprising the following steps:
(a) delivering to the esophagus of a subject, e.g. a human or non-human animal, an esophageal stent as herein described;
(b) waiting for a time period necessary for the photosensitising agent to achieve an effective tissue concentration at the desired site in the esophagus;
(c) optionally removing the stent; and
(d) photoactivating the photosensitising agent.

1-15. (canceled)

16. An esophageal stent comprising a generally cylindrical stent body having a surface coating comprising an active agent which is a photosensitising agent or a precursor thereof.

17. The stent as claimed in claim 16, wherein said stent body is made of metal, or of a polymer.

18. The stent as claimed in claim 16 wherein said coating comprises two or more coating layers, at least one of which comprises said active agent.

19. The stent as claimed in claim 16, wherein said coating is provided in the form of a powder, a cake or a film.

20. The stent as claimed in claim 16, wherein said active agent is selected from the group consisting of hematoporphyrin derivative, hematoporphyrines, Photosan III, chlorins and their bacteriochlorins, mono-L-aspartyl chlorin e6, chlorin e6, benzoporphyrins, purpurines, phthalocyanines, porphycenes, hypocrellins, protoporphyrin IX, hematoporphyrin di-ethers, uroporphyrins, coproporphyrins, deuteroporphyrin, polyhematoporphyrin, lutetium texaphyrin 5-ALA and esters of 5-ALA, porphobilinogen and precursors and derivatives thereof.

21. The stent as claimed in claim 16, wherein said active agent is 5-ALA or a precursor or derivative of 5-ALA.

22. The stent as claimed in claim 21, wherein said active agent is a 5-ALA ester or a pharmaceutically acceptable salt thereof.

23. The stent as claimed in claim 22, wherein said active agent is a 5-ALA ester of formula I or a pharmaceutically acceptable salt thereof:
R²₂N—CH₂COCH₂—CH₂CO—OR¹ (I)
wherein
R¹ represents a substituted or unsubstituted alkyl group; and
R² each independently represents a hydrogen atom or a group R¹.

24. The stent as claimed in claim 23, wherein said 5-ALA ester of formula I is provided in the form of a pharmaceutically acceptable salt.

25. The stent as claimed in claim 24, wherein said salt is selected from the group consisting of HCl salt, nitric acid salt, sulfonic acid salt and sulfonic acid derivative salt.

26. The stent as claimed in claim 22, wherein the 5-ALA ester is 5-ALA hexyl ester.

27. The stent as claimed in claim 16, wherein said coating comprises one or more polymers which have good film-forming properties and/or good gel-forming properties.

28. The stent as claimed in claim 27, wherein said one or more polymers are selected from the group consisting of cellulose ethers, gellan gum, chitosan, chitosan derivatives, pullulan, alginates, hyaluronic acid, hyaluronic acid derivatives and carrageenan.

29. The stent as claimed in claim 16, wherein said coating further comprises one or more other pharmaceutically acceptable excipients.

30. The stent as claimed in claim 29, wherein said one or more other pharmaceutically acceptable excipients are selected from the group consisting of plasticizers, coloring agents, thickening agents, disintegrants, mucoadhesive agents, surface penetration assisting agents and chelating agents.

31. A method for preparing the stent as claimed in claim 16, said method comprising the following steps:
(a) providing a generally cylindrical stent body; and
(b) applying to said body a coating comprising an active agent which is a photosensitising agent or a precursor thereof.

32. The method as claimed in claim 31, wherein said coating is applied by a film coating process, by solvent evaporation or by lyophilization.

33. The method as claimed in claim 32 wherein said coating is applied by a film coating process selected from the group consisting of dip coating and spray coating.

34. A method of diagnosis of a portion of the epithelial surface of an esophagus of a human patient, said method comprising the following steps:
(a) delivering to the esophagus of said patient an esophageal stent comprising a generally cylindrical stent body having a surface coating comprising an active agent which is a photosensitising agent or a precursor thereof;
(b) waiting for a time period necessary for the photosensitising agent to achieve an effective tissue concentration at the desired site in the esophagus;
(c) optionally removing the stent;
(d) photoactivating the photosensitising agent;
(e) detecting fluorescence from said photosensitising agent using a fluorescence detector; and
(f) optionally converting the detected fluorescence into an image of an area of interest in the surface of the esophagus.

35. A method of treating an abnormality of an epithelial-lined surface of an esophagus in a human patient, said method comprising the following steps:
(a) delivering to the esophagus of the patient an esophageal stent comprising a generally cylindrical stent body having a surface coating comprising an active agent which is a photosensitising agent or a precursor thereof;
(b) waiting for a time period necessary for the photosensitising agent to achieve an effective tissue concentration at the desired site in the esophagus;
(c) optionally removing the stent; and
(d) photoactivating the photosensitising agent.
